(19) United States
(12) Patent Application Publication
(10) Pub. No.: US 2007/0275001 A1
(43) Pub. Date: Nov. 29, 2007

(54) Rational Design and Engineering of Proteins and Peptides for Immunomodulation

(75) Inventors: Lokesh Joshi, Tempe, AZ (US); Giovanna Ghirlanda, Scottsdale, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Arizona Board of Regents

(21) Appl. No.: 10/588,431

(22) PCT Filed: Feb. 7, 2005
(86) PCT No.: PCT/US05/04043

§ 371 Date: May 21, 2007

Related U.S. Application Data
(60) Provisional application No. 60/542,117, filed on Feb. 5, 2004.

(51) Int. Cl. A61K 39/00; A61P 35/00; A61P 37/04; C07K 14/52; C07K 7/06

(52) U.S. Cl. 424/185.1; 530/324; 530/328

(57) ABSTRACT

The present invention discloses an immunomodulatory protein or peptide mimetic and method for treatment of immunosuppressive diseases and conditions by administering an effective dose of the immunoactive form of the mimetic sufficient to activate phagocytic cells and triggering phagocytosis, thereby activating the immune system.

### CLAIM TO DOMESTIC PRIORITY

This Application claims the benefit of priority of U.S. Patent Application Ser. No. 60/542,117, filed Feb. 5, 2004.

### STATEMENT REGARDING FEDERALLY SPONSORED RESEARCH

### FIELD OF THE INVENTION

The present invention relates to a treatment for immunosuppressive diseases and conditions, and more specifically to protein and peptide mimetics that activate phagocytic cells of the immune system and trigger phagocytosis.

### BACKGROUND OF THE INVENTION

The immune system represents the endogenous defense mechanism that constantly scans for ‘self’ and ‘non-self’ molecules and organisms in the body. The immune response against ‘non-self’ entities is initiated upon theirs encounter with the phagocytic cells, such as macrophages and dendritic cells. The phagocytic cells engulf and digest the foreign substance/cells and display specific antigens on their surface. These antigenic fragments alert a specific type of T lymphocyte, the helper T cell, to begin a precisely choreographed attack that ultimately results in cellular and humoral immunity against the foreign intruder.

The immune system also recognizes cancer cells as foreign and removes them. However, the fact that cancer cells manage to escape this surveillance suggests that either the immune system does not easily distinguish cancer cells from healthy cells and/or cancer suppresses the immune system. Several approaches have been used to stimulate, repair or enhance the immune system, such as specific antibody targeting, lymphokine treatment and infusion of activated dendritic cells. The induction of tumor immunity can be initiated by the effectors of innate immunity and further developed by cells of adaptive immunity, with phagocytic cells such as macrophages and dendritic cells playing a central role in linking these defense mechanisms.

This cycle can be initiated by many bacterial cell wall constituents, such as lipopolysaccharide (LPS), lipid A, muramyl peptides, and their derivatives. Although LPS is a very potent activator of the immune response, its toxicity prevents its use in therapy. Synthetic oligodeoxynucleotides containing CG motifs (CpG ODNs) have been shown to have potent immunostimulatory properties and have been proposed as effective vaccine adjuvants. Many oligosaccharides and glycoproteins have also been implicated in immune modulation. Some of these compounds have been applied clinically as adjuvant in cancer treatment, for example, beta-(1-3)-linked D-glucans that are found as constituents of fungi, algae and higher plants.

Biologic response modifications with immune stimulators, lymphokines, antibodies or specific carbohydrate epitopes activate the immune system to recognize cancer cells. Vitamin D Binding Protein (VDBP or Gc-globulin) is a multi-functional serum glycoprotein. VDBP is the precursor of Gc-MAF, an evolutionarily conserved polymorphic serum glycoprotein composed of three distinct domains (FIG. 1A). The most common forms of this protein are Gc1F, Gc1S and Gc2 which differ slightly in amino acid composition and glycosylation states.

VDBP is converted to Macrophage Activating Factor (Gc-MAF) by post-translation modifications. A single N-acetyl-galactosamine (GalNAc) mediates the interaction of Gc-MAF with a receptor on the macrophage surface. This interaction results in macrophage activation for phagocytosis and antigen presentation.

Macrophage activating factor (Gc-MAF) is an abundant serum glycoprotein composed of three domains. The C terminal domain III contains 120 amino acids and is crucial for macrophage activation. Domain III of precursor Gc-MAF is post-translationally O-glycosylated at threonine 420 with an oligosaccharide moiety composed primarily of N-acetyl-D-galactosamine (GalNAc), galactose and sialic acid residues Activation of Gc-MAF is accomplished by selective removal of sugars by galactosidase and sialidase present on B- and T-cells, respectively (FIG. 1B). A single GalNAc residue is retained, and mediates the interaction of activated Gc-MAF with a receptor on the macrophage surface. This interaction results in macrophage activation for phagocytosis and subsequent antigen presentation.

The product Gc-MAF putatively activates macrophages through an interaction of the GalNAc residue with a receptor on the macrophage surface. In a recent report, similar lectins have been described on monocyte-derived dendritic cells, supporting the high likelihood of dendritic cell activation by Gc-MAF. Extensive work by Yamamoto and colleagues (Yamamoto and Kumashiro, 1993; Yamamoto and Naraparaju, 1996 a,b) suggested that DBP has remarkable therapeutic value as an activator of macrophages. The active form of the protein reduces tumor cell load (Kisker et al., 2003; Onizuka et al., 2004), provides a therapy against viral infections such as HIV (Yamamoto et al., 1995), promotes bone growth (Schneider et al., 1995; 2003) and therapy against bone disorders such as ostepetrosis (Yamamoto et al., 1996b), has been found to be an effective anti-angiogenesis factor (Kanda et al., 2002; Kisker et al., 2003), and is a potent adjuvant for immunizations (Yamamoto and Naraparaju, 1998).

However, cancerous cells secrete a-N-acetyl-D-galactosaminidase (GalNAcase) into the blood stream, which results in complete deglycosylation of serum Gc-MAF leading to immunosuppression. It has been shown that the administration of enzymatically activated Gc-MAF to Ehrlich ascite tumor-bearing mice will overcome the inactivation and result in macrophage activation in less than 6 hr. Injection of Gc-MAF also substantially increases initiation of antibody production within 48 hr. These observations show that Gc-MAF can be useful as an adjuvant to enhance and accelerate the development of the immune response and to generate a large amount of antigen-specific antibodies.

Until recently, mammalian serum has been the only available source of Gc-MAF, restricting is applications to patients. The use of mammalian blood-derived proteins for therapeutic applications causes a real concern of disease transmission from contaminating viruses, prions and other infectious agents within animal systems. Moreover, the native protein has other biological functions, such as the transport of vitamin D and a role in the removal of actin from serum. The administration of exogenous protein in large quantities could potentially interfere with known and unknown activities of the protein, leading to unforeseen collateral effects.

In recent years, rational protein design has proved to be a valuable tool for optimizing therapeutic proteins. Several engineered proteins obtained by rational design are currently on the market or have completed clinical trials, generating a revenue of approximately US$30 billions in 2001. Examples of engineered protein therapeutics are HumaLog® (Eli Lilly) and NovoLog® (Novo Nordisk), fast-acting versions of insulin; Ontak® (Seragen), a natural toxin reengineered to target cancer cells; Fuzeon® (Trimeris), an inhibitor of HIV fusion derived from the viral protein gp41.

In these drugs, properties such as activity, stability, solubility, specificity, immunogenicity and pharmacokinetics have been successfully optimized (FIG. 2). Starting from the detailed knowledge of the protein structure, rational design involves computational simulations and evaluations of mutants that are ultimately screened for activity in vitro and in vivo.

The experimental data obtained on each mutant can be utilized for the design of second-generation optimized proteins. This last step is conceptually similar to traditional Quantitative Structure-Activity Relationships (QSAR) methods, but utilizes protein-specific computational methods.

Domain III of Gc-MAF is the site of the specific glycosylation event that leads to its bioactivation. In broader terms, a domain of a protein is an independently folded unit that can be separated from the intact protein and retain a specific structure. Domains often serve as the smallest functional elements of a complex protein, in which two or more domains can be combined to obtain complex functions. For example, Gc-MAF contains a vitamin D-binding domain, an actin-binding domain, and the glycosylation site, Domain III, which is crucial for macrophage activation.

The use of Domain III in lieu of full length Gc-MAF in therapy would present several advantages: first, the size of the isolated Domain III, ca. 120 amino acids, would make it a more tractable drug; second, by dissecting the desired function one would avoid possible cross-reactivity and side effects. However, preliminary studies show that the activity of the isolated Domain III is significantly reduced in comparison to the holo-Gc-MAF activity. A possible explanation is given by visual inspection of the crystal structure of Gc-MAF. Domain III is a distorted three-helix bundle, and makes significant hydrophobic and ionic contacts with the remaining Gc-MAF (FIG. 3). Thus, the isolated domain would not be sufficiently stable under physiological conditions.

Therefore, a need exists for a treatment of immunosuppressive diseases and conditions comprised of a physiologically stable protein or peptide mimetic that exhibits the immunomodulatory activity of Gc-MAF that is easily tractable, yet limits cross-reactivity and side effects.

### BRIEF DESCRIPTION OF THE FIGURES

FIG. 1A is the amino acid sequence, predicted secondary structure features and the C-terminal O-glycosylation site (arrow) of Vitamin D-binding protein.

FIG. 1B is a schematic diagram showing the in vivo activation of MAF by selective deglycosylation by β-galactosidase and sialidase and its inactivation by N-acetyl galactosaminidase.

FIG. 2 illustrates strategies for rational design and protein engineering.

FIG. 3 shows the crystal structure of MAF. Domain III is bold.

FIG. 4 is a ribbon representation of Domain III (A), the scaffold 1LQ7 (B), superimposition of the loop of Domain III onto one of the loops of 1LQ7 (C), and superimposition onto both loops of 1LQ7 (D).

FIG. 5 is a molecular model of the glycosylated MM1.

FIG. 6 is a MALDI-TOF spectra of A) MM1 and B) Gc-MM1. In both cases, the low mass species is the double charged.

FIG. 7 is a CD spectrum (A) of MM1 and Gc-MM1; both proteins are highly helical. (B) Equilibrium chemical denaturation curve of MM1.

FIG. 8 illustrates the quantification of phagocytosis by flow cytometry. A) non stimulated control cells; B) cells stimulated with Gc-MAF; C) cells stimulated with Gc-MM1.

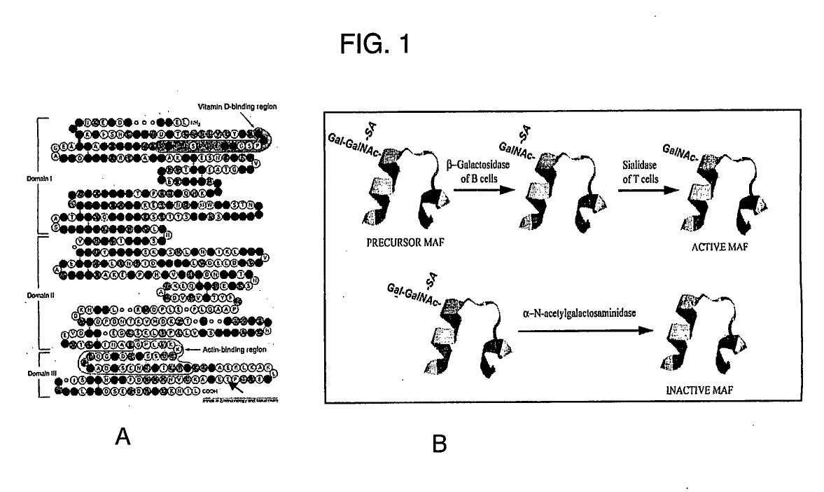

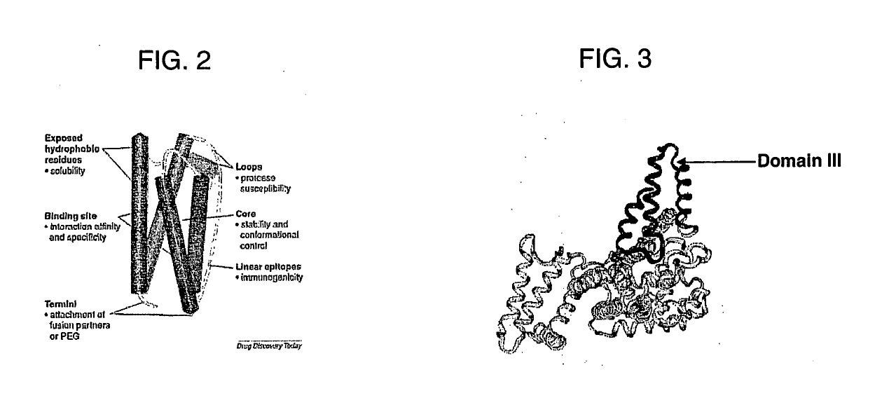

### DETAILED DESCRIPTION

Cancer is one of the leading causes of death. A serum protein macrophage activating factor (Gc-MAF), stimulates phagocytic immune cells to identify, ingest and digest cancer cells and/or other foreign particles. Until now, the blood serum of the mammalian systems has been the primary source of this therapeutic protein.

As noted above, the use of mammalian blood-derived proteins for therapeutic applications causes a real concern of disease transmission from contaminating viruses, prions and other infectious agents within animal systems. Moreover, the native protein has other biological functions, such as the transport of vitamin D and a role in the removal of actin from serum. The administration of exogenous protein in large quantities could potentially interfere with known and unknown activities of the protein, leading to unforeseen collateral effects.

The present invention circumvents these problems by using molecular modeling and protein engineering technology to isolate the putative active site of Gc-MAF and display it on an artificial protein scaffold, with the aim of developing smaller mini-protein analogs of the immunomodulatory macrophage activating factor protein. This process produces structurally and functionally optimized mimics of Gc-MAF as an effective adjuvant to immunotherapy of cancer and other diseases. This represents a novel approach that has significant therapeutic and biotechnological potential.

The rational design and engineering of biologically active mini-protein mimics of naturally occurring proteins disclosed herein are of significant medical importance. Molecular modeling and protein engineering enables the synthesis of biomolecules with optimal structural and biological properties. The ultimate result of this invention is a protein-based therapeutic platform technology to treat human diseases and specifically develop protein/peptide based therapeutic agents to treat/prevent cancer and other immune related diseases.

Rational Design of a Gc-MAF Analog

The present invention uses a rational design approach to prepare optimized, miniaturized proteins as mimetics of Domain III. Because of the distorted three-helix bundle topology of Domain III, a stable peptide scaffold of similar but more regular topology is needed as a starting point. According to the present invention, the putative active site of Domain III, defined as the portion of the protein surrounding the glycosylation site, was grafted onto a stable three-helix bundle scaffold obtained form the Protein Data Bank and the resulting model protein was optimized as described below.

The choice of the scaffolds was guided by three considerations: first, the size is considerably smaller than Domain III, and well within the limits for solid-phase synthesis of peptides; second, the scaffold is well characterized in terms of its stability and biophysical properties; third, the scaffold is amenable to structural studies. This approach has the advantage of starting from a structured template with minimal sequence homology to the native protein, thus avoiding possible interferences with undesired functions of the native protein. The increased stability of the analog will minimize its sensitivity to proteases, which would be an important consideration in the in vivo use of biomimetics. Moreover, the prototype protein, MM1, can be optimized by computer modeling and rational mutagenesis.

In order to design an optimized version of Domain III, computer graphics to identify the critical residues for activity were used. Specifically, the glycosylated threonin (Thr) 420 is located in a solvent-exposed loop and protrudes from the start of one of the helices; the sequence surrounding Thr 420 is likely recognized by glycosylation enzymes and by the specific receptor located on the surface of the macrophages. Thus, the putative active sequence was narrowed to approximately 20 residues, which comprise the loop and the first turn of the α-helix on each side.

Using Insight II, a molecular modeling software package, the stability of the isolated 20 amino acid sequence was determined by running energy minimization experiments. The results show that the minimized loop presents severe deviations from the three-dimensional structure assumed in the native protein. Clearly, the underlying three-helix structure of Domain III is critical to restrain the conformation of the loop to the biologically active form. Therefore, the active loop of Domain III in the native conformation was transferred onto a more stable three-helix bundle obtained from the Protein Data Bank.

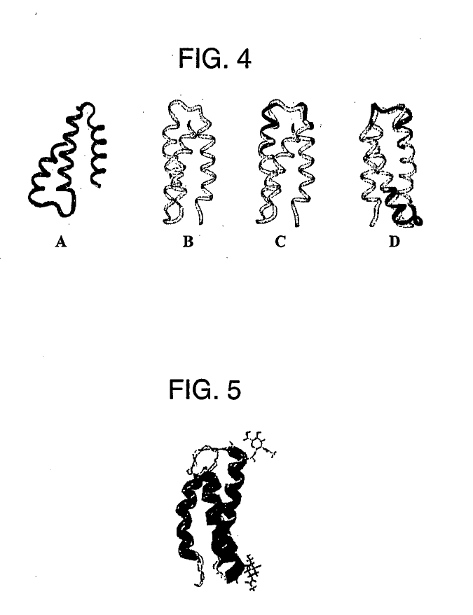

The scaffold chosen, 1LQ7, was originally designed at the University of Pennsylvania and has the additional advantage of being amenable to solid-state synthesis. The sequence of the 20 amino acid loop was aligned with that of the scaffold, using the position of the helical residues on each side of the loop as guide. The aligned coordinates of the loop was overlaid onto those of the template (FIG. 4), obtaining a remarkable superimposition of the two structures.

The scaffold loop was then replaced with that of the Domain III; a few alternative fragment lengths were tested for the substitution. In order to increase the protein activity, both scaffold loops were replaced with Domain III amino acids to yield a bifunctional molecule. The miniaturized proteins retained the overall three-helix bundle topology of Domain III, in a more regular and stable version. Each version was optimized by energy minimization routines and evaluated to identify significant deviations from the native loop conformation. The model that showed the smallest deviations from the native conformation was selected and will be the starting point for protein optimization.

TABLE 1
Sequence comparison:

| | | | | |
|---|---|---|---|---|
| Helix 1 | Loop1 | Helix 2 | Loop2 | Helix 3 |

| | | | | | |
|---|---|---|---|---|---|
| 1LQ7 | GSRVKALEEKVKALEEKVKAL | GGGG | RIEELKKKWEELKKKIEEL | GGGG | EVKKVEEEVKKLEEEIKKL |
| MM1 | GSRVKALEEKVKALEEKVKAL | GNAT | PTELAKKKWEELKKKIEEL | GNATPT | EVKKVEEEVKKLEEEIKKL |

As shown in Table 1, the final sequence differs from that of the scaffold by 10 mutations, corresponding to the loop regions. The minimized model was modified by attaching GalNAc residues to the second threonine in each loop, corresponding to the glycosylated Thr 420 of Domain III (FIG. 5).

The putative active site spans the four residues in the loop, as well as the first half turn in the helix. In Loop 1, the superimposition with the scaffold required only changing the residue composition. In Loop 2, the superimposition required the addition of two additional residues, basically elongating helix 3 by a little bit at the N terminal. In order to have nondisruptive mutations, the hydrophobic residues in Gc-MAF had to be aligned, beyond the part used for the copy and paste, to make sure that the loops and the helices were in register.

As shown in Tables 2 and 3, several amino acid sequences may be used for the Loop 1 and/or Loop 2 portions of MM1 in synthesizing an immunomodulatory protein according to the present invention. Additionally, it is disclosed that other scaffolds, in addition to 1LQ7 may be used with the Loop 1 and/or Loop 2 positions, as long as the putative active site sequence is maintained.

Thus, Table 2 comprises a non-limiting list of amino acid sequences for each putative active site at Loop 1 and Loop 2, respectively, that will preserve the activity of each putative site. It is further disclosed that the synthesized protein or peptide need only have the presence of one of the Loop 1 or Loop 2 sequence in order to be an effective immunomodulatory treatment.

As is shown in Tables 2 and 3, the Loop 1 putative active site sequence spans Residues 22 through 31. Each column illustrates the possible amino acids that can be used at each residue position in the synthesized protein according to the present invention.

TABLE 2
Loop 1 Sequences

| Res 22 | Res 23 | Res 24 | Res 25 | Res 26 | Res 27 | Res 28 | Res 29 | Res 30 | Res 31 |
|---|---|---|---|---|---|---|---|---|---|
| G | N | A | T | P | T | E | L | A | K |
| P | D | G | N | G | S | N | A | L | R |
| A | E | L | S | | K | Q | V | V | D |
| N | G | V | K | | E | D | I | I | E |
| S | | F | E | | N | K | F | F | N |
| | | | D | | D | R | W | W | Q |
| | | | Q | | Q | | Y | Q | |
| | | | | | | | | N | |

As is shown in Table 3, the Loop 2 putative active site sequence spans Residues 45 through 54. Each column illustrates the possible amino acids that can be used at each residue position in the synthesized protein according to the present invention.

TABLE 3
Loop 2 Sequences

| Res 45 | Res 46 | Res 47 | Res 48 | Res 49 | Res 50 | Res 51 | Res 52 | Res 53 | Res 54 |
|---|---|---|---|---|---|---|---|---|---|
| G | N | A | T | P | T | E | V | K | K |
| P | D | G | N | G | S | N | A | R | R |
| A | E | L | S | | K | Q | L | D | D |
| N | G | V | K | | E | D | I | E | E |
| S | | F | E | | N | K | F | N | N |
| | | | D | | D | R | W | Q | Q |
| | | | Q | | Q | | Y | A | |

Synthesis and Purification

The putative active sequence of Domain III, which comprises the glycosylated loop and the first turn of the α-helix on each side, was used to replace both scaffold loops and a few alternative fragment lengths were tested for the substitution. Energy minimization routines using the module Discover (Biosym) allowed to choose the best model as the one with the smallest deviations from the native conformation. The resulting 69 residue model peptide was called glycosylated Mini MAF1 (Gc-MM1). The non-glycosylated analog (MM1) was also prepared as negative control for the biophysical characterization and the activity screening.

The MM1 peptide was synthesized on a Milligen 9050 automated peptide synthesizer using PAL resin on a 0.2 mmol scale using Fmoc-protection solid phase methodology. Unreacted chains were capped by acetylation at each step of the synthesis to prevent further reactions. The N-terminal was also acetylated after completion of the synthesis. After cleavage from the resin with TFA, the peptide has a C-terminal amide group. The solid peptide was dried and purified by reverse phase HPLC on a semipreparative Vydac C-4 column using a linear gradient of water and acetonitrile containing 0.1% of TFA. The N terminus is acetylated, and the C terminus is amidated in MM1. It is disclosed that peptides with no modifications at the termini, or with different modifications (e.g., PEG, amines, esters) will also be active.

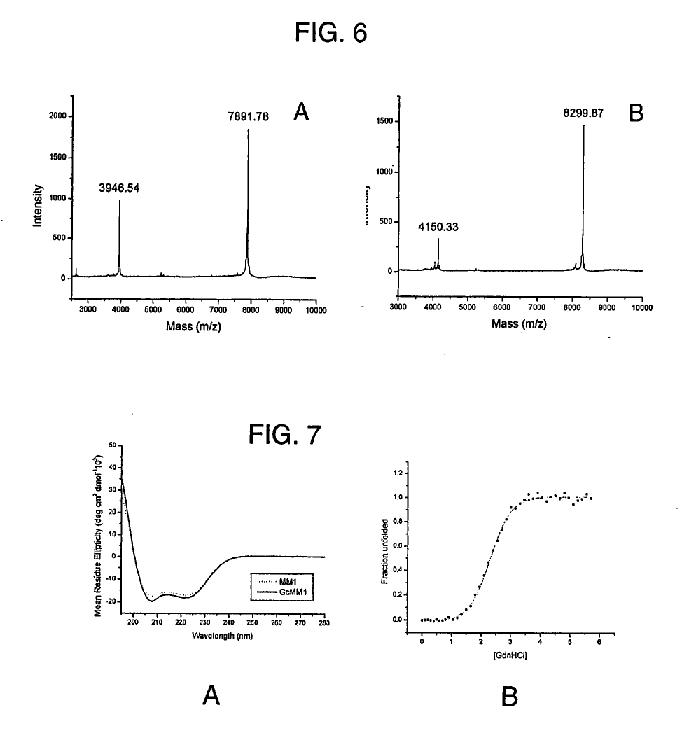

For the glycosylated peptide, an additional step was necessary to remove the protective acetyl groups from the N-acetyl-galactosamine residues. Purified Gc-MM1 was treated with a solution of 130 mM sodium methoxide in methanol for 5 hr. at room temperature. The molecular mass of pure MM1 and Gc-MM1 was then confirmed with matrix-assisted laser desorption mass spectrometry (MALDI-TOF) (FIG. 6). Analytical equilibrium sedimentation ultracentrifugation confirmed that the protein exists as a monomer in solution. The main product, when analyzed by MALDI, confirmed the expected molecular weight (7892 Da).

As described above, the present invention discloses both glycosylated and non-glycosylated mimetics. In the natural MAF protein, sugars (glycans) are attached to the threonine (T) residue. In the mimetic, as disclosed herein, any sugar from the hexose or hexosamine groups may be attached to the threonine in the glycosylated form of the mimetic. As shown in Tables 2 and 3, in the mimetic, the threonine residue can be also be substituted with several other amino acids, for example, asparagines and serine, which then substitute as the glycosylated site(s). Further, in the mimetic, the amino acid sequence can be varied to structurally represent the glycan moiety.

Characterization of Gc-MAF Domain III-Analog

The physical and chemical properties of protein therapeutics are critical factors that influence the ease of manufacturing, development and clinical use. The Gc-MAF-mimic was evaluated in terms of its solubility, aggregation state and stability using a variety of biophysical methods. The secondary structure of the proteins was determined by circular dichroism (CD) spectroscopy: the far-UV spectrum of MA1 in aqueous buffer shows the minima at 208 and 222 nm characteristic of a α-helical conformation (FIG. 7A).

The measurements were carried on a Jasco J-710 spectropolarimeter with cell holder temperature controlled at 25° C. The bandwidth was 1.00 nm. Peptides were dissolved in 10 mM phosphate buffer, pH 7.0. Protein concentrations were 2 μM and 19 μM respectively, as determined using tryptophan absorbance, taking ε₂₈₀=5700 M⁻¹·cm⁻¹. CD intensity is expressed as mean residue ellipticity, [Θ], given by [Θ]=[Θ]_{obs}/10/Cn where [Θ]_{obs} is the observed ellipticity in degrees, l is the cuvette path length in centimeter, C is the molar concentration; n represents the number of amino acids. The mean residue ellipticity at 222 nm ([Θ]₂₂₂ ) is −24.9·10³ deg cm² dmol⁻¹, and the ratio between the mean residue ellipticities at 222 nm and 208 nm ([Θ]_{222/}[Θ]₂₀₈) is 0.96; these values are consistent with a highly helical structure, accounting for three helices of approximately 20 residues each.

The thermodynamic stability of the proteins was assessed by chemical denaturation studies, in which the CD signal at 222 nm was monitored at increasing concentrations of denaturant agent, guanidinium hydrochloride (FIG. 7B). A 1 cm path length rectangular quartz cell was used. The cell holder was temperature controlled at 25° C. The buffer was 10 mM potassium phosphate, pH 7.0. The bandwidth was 1.00 nm. At each GdnHCl concentration, cell chamber was equilibrated for 6 minutes, then data were collected. The curve is described by the equation: ΔG_{obs}=ΔG_{H2O}+m[GdnHCl] in which ΔG_{obs} is the free energy for the two state unfolding equilibrium observed at a given concentration of GdnHCl, ΔG_{H2O} is the free energy of denaturation extrapolated to zero GdnHCl concentration, m is a constant that provide a measure of the cooperativity of the process.

The resulting sigmoidal curve was analyzed to extrapolate the free energy of folding, ΔG, estimated to be −4.2 Kcal/mol; the corresponding ΔG for 1LQ7 is −4.6 Kcal/mol. The content of helical structure at room temperature and the free energy of folding are independent of the concentration, indicating that the designed peptide is monomeric.

This finding was corroborated by equilibrium sedimentation analysis, performed using a Uv-Vis monitored analytical centrifuge, which yielded an apparent molecular weight in solution of 7900 Da for the non-glycosylated MM1. More importantly, the free energy of folding is within 30% of that of the original scaffold protein, 1LQ7. The thermodynamic analysis is in agreement with the molecular dynamics studies, showing that the core helical bundle of MM1 is identical to that of 1LQ7; only the spliced loops deviate appreciably from the position occupied in the scaffold protein.

These data indicate that the spliced loop was well tolerated by the three-helix bundle and that the prototype Gc-MAF analog is of comparable stability to natural proteins of similar length.

Biological Activity

Rapid, Quantitative in vitro Test for Macrophage Activation

The present invention discloses a method for rapid, quantitative in vitro testing for macrophage activation. Phagocytosis is a cytoskeleton-dependent process of engulfment of large particles. Phagocytes use various surface receptors to bind and internalize the foreign particles for processing the pathogens in lysosomes (phagolysosomes) for presentation of antigens to the immune system. The effects of Gc-MAF with both positive and negative controls of phagocytosis are determined. As positive controls, macrophages are also stimulated with Beta-1,3 glucans, a well-known immune stimulator, for functional comparison. Specifically, curdlan, linear (1,3)-Beta-D-glucans are used. The opsonized FITC labeled latex beads are used as the tracer of phagocytosis in this cultured macrophage model. The ingestion of biotynilated mouse IgG Bound to streptavidin coated FITC labeled latex beads was used for the phagocytosis assay.

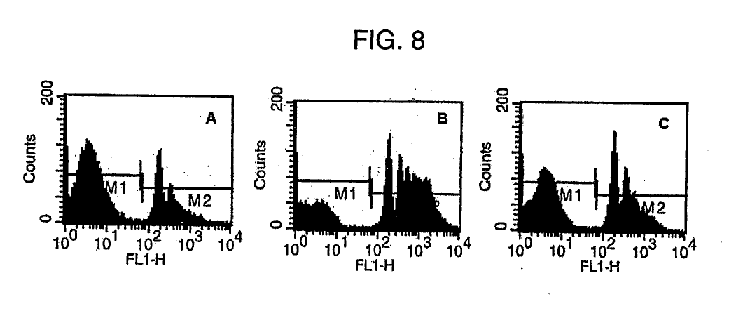

Once the cells are suspended in the culture medium, quantification of phagocytosis is accomplished by flow cytometry. One unique feature of flow cytometry is that it measures fluorescence per cell or particle. FIG. 8 illustrates the quantification of phagocytosis by flow cytometry. A) non-stimulated control cells; B) cells stimulated with Gc-MAF; C) cells stimulated with Gc-MM1.

Here, the cells are stimulated overnight and then incubated with FITC labeled beads conjugated with IgG for 30′. Cells are then washed twice with PBS, detached mechanically and resuspended in PBS with 1% BSA, 0.05% Triton X-100 for FACS analysis. A FACSCalibur (FACS=Fluorescence Activated Cell Sorter) system (Becton Dickinson) equipped with an air-cooled argon ion laser (488 nm, 15 mW output) is used for this study. Forward light scatter (FSC) and 90° light scatter (SSC) were measured at 488 nm and fluorescence emissions (FL parameters) were collected using the FSC as the triggering signal. Fluorescence data was reported by CellQuest software (Becton Dickinson).

FITC fluorescence signals were measured on FL1 channel (564-606 nm). A total of 30,000 events were recorded for each sample. Markers M1, between 10° and 10², and M2, between 10² and 10⁴, were determined (FIG. 8). M2 values define the percentage of cells considered as positive to the ingestion. Control, non-stimulated cells showed 43.6±10.3% of cells positive to phagocytosis with a mean fluorescence intensity of 543±149; whereas cells stimulated with Gc-MAF and Gc-MM1 showed 57.5±6.6% and 48.1±3.6 of positive cells with a mean fluorescence of 724±155 and 598±168 respectively.

The present invention provides methods for stimulating immune system activity in a subject, comprising administering to a subject an amount effective of a protein according to the invention for stimulating immune system activity. As used herein the phrase “stimulating immune system activity” means to increase the activity of one or more components of the immune system, including phagocytes, macrophages, and neutrophils. Substances secreted by activated macrophages in turn stimulate other cells of the immune system, in particular dendritic cells. As such, methods for stimulating immune system activity are broadly useful for treating cancer, viral infections, angiogenesis-mediated disorders, bone disorders, immune-suppressed disorders, pain, and as adjuvants for vaccinations.

The present invention further provides methods for treating one or more disorders in a subject, selected from the group consisting of viral infection, cancer, bone disorders, immune suppressed disorder, pain, and angiogenesis-mediated disorders, comprising administering to a subject an amount effective of a protein according to the invention for treating the disorder.

The present invention further provides methods for promoting an improved immune system response to a vaccination, comprising administering to a subject receiving a vaccination an amount effective of a protein according to the invention for promoting an improved immune system response to the vaccination. In carrying out the methods for promoting an improved immune system response to the vaccination according to the present invention, the proteins, or pharmaceutical compositions thereof, of the invention can be administered before, simultaneously with, or after vaccine administration. Where the vaccine is administered on multiple occasions, the proteins of the invention can be administered together with a single vaccine administration, or with multiple vaccine administrations. In a preferred embodiment, the proteins are administered simultaneously with the one or more rounds of vaccination. Preferred classes of patients include populations at high risk for viral infection, including but not limited to children, health care workers, senior citizens, and those at high risk of specific types of viral infection, such as partners of HIV infected individuals, sex trade workers, and intravenous drug users.

In a preferred embodiment of the methods of the invention, the subject is a mammal; in a more preferred embodiment, the subject is a human.

In various embodiments of the methods of the invention, administration of the protein is accomplished via direct delivery (for example, by injection), or by gene therapy via administration of an appropriate expression vector of the invention which can be expressed in the target tissue. In embodiments employing gene therapy, it is preferred to use viral expression vectors, including but not limited to adenoviral and retroviral vectors.

In carrying out the methods of the invention, the proteins or pharmaceutical compositions thereof may be made up in a solid form (including granules, powders, transdermal or transmucosal patches or suppositories) or in a liquid form (e.g., solutions, suspensions, or emulsions), and may be subjected to conventional pharmaceutical operations such as sterilization and/or may contain conventional adjuvants, such as stabilizers, wetting agents, emulsifiers, preservatives, cosolvents, suspending agents, viscosity enhancing agents, ionic strength and osmolality adjustors and other excipients in addition to buffering agents. Suitable water soluble preservatives which may be employed in the drug delivery vehicle include sodium bisulfite, sodium thiosulfate, ascorbate, benzalkonium chloride, chlorobutanol, thimerosal, phenylmercuric borate, parabens, benzyl alcohol, phenylethanol or antioxidants such as Vitamin E and tocopherol and chelators such as EDTA and EGTA. These agents may be present, generally, in amounts of about 0.001% to about 5% by weight and, preferably, in the amount of about 0.01 to about 2% by weight.

For administration, the proteins are ordinarily combined with one or more adjuvants appropriate for the indicated route of administration. The proteins may be admixed with alum, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the proteins of this invention may be dissolved in physiological saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

For use herein, the proteins may be administered by any suitable route, including local delivery, parentally, transdermally, by inhalation, or topically in dosage unit formulations containing conventional pharmaceutically acceptable carriers, adjuvants, and vehicles. The term parenteral as used herein includes, subcutaneous, intravenous, intramuscular, intrasternal, intratendinous, intraspinal, intracranial, intrathoracic, infusion techniques or intraperitoneally. Suppositories for rectal administration of the active agents in combination with the vaccines can be prepared by mixing the drug with a suitable non-irritating excipient such as cocoa butter and polyethylene glycols which are solid at ordinary temperatures, but liquid at the rectal temperature and will therefore melt in the rectum and release the drug.

Solid dosage forms for oral administration may include capsules, tablets, pills, powders and granules. In such solid dosage forms, the proteins may be admixed with at least one inert diluent such as alum, sucrose, lactose or starch. Such dosage forms may also comprise, as is normal practice, additional substances other than inert diluents, e.g., lubricating agents such as magnesium stearate. In the case of capsules, tablets and pills, the dosage forms may also comprise buffering agents. Tablets and pills can additionally be prepared with enteric coatings. Liquid dosage forms for oral administration may include pharmaceutically acceptable emulsions, solutions, suspensions, syrups and elixirs containing inert diluents commonly used in the art, such as water. Such compositions may also comprise adjuvants, such as wetting agents, emulsifying and suspending agents and sweetening, flavoring and perfuming agents.

As used herein for all of the methods of the invention, an “amount effective” of the proteins is an amount that is sufficient to provide the intended benefit of treatment. An effective amount of the proteins that can be employed ranges generally between about 0.01 μg/kg body weight and about 10 mg/kg body weight, preferably ranging between about 0.05 μg/kg and about 5 mg/kg body weight. However, dosage levels are based on a variety of factors, including the type of disorder, the age, weight, sex, medical condition of the individual, the severity of the condition, the route of administration, and the particular compound employed. Thus, the dosage regimen may vary widely, but can be determined routinely by a physician using standard methods.

Tumors susceptible of treatment by the methods of the invention include lymphomas, sarcomas, melanomas, neuroblastomas, carcinomas, leukemias, and mesotheliomas. Methods of tumor treatment according to the invention can be used in combination with surgery on the subject, wherein surgery includes primary surgery for removing one or more tumors, secondary cytoreductive surgery, and palliative secondary surgery. In a further embodiment, the methods further comprise treating the subject with chemotherapy and/or radiation therapy, which can reduce the chemotherapy and/or radiation dosage necessary to inhibit tumor growth and/or metastasis. As used herein, “radiotherapy” includes but is not limited to the use of radio-labeled compounds targeting tumor cells. Any reduction in chemotherapeutic or radiation dosage benefits the patient by resulting in fewer and decreased side effects relative to standard chemotherapy and/or radiation therapy treatment. In this embodiment, the polypeptide may be administered prior to, at the time of, or shortly after a given round of treatment with chemotherapeutic and/or radiation therapy. In a preferred embodiment, the protein is administered prior to or simultaneously with a given round of chemotherapy and/or radiation therapy. In a most preferred embodiment, the protein is administered prior to or simultaneously with each round of chemotherapy and/or radiation therapy. The exact timing of compound administration will be determined by an attending physician based on a number of factors, but the polypeptide is generally administered between 24 hours before a given round of chemotherapy and/or radiation therapy and simultaneously with a given round of chemotherapy and/or radiation therapy. The tumor treating methods of the invention are appropriate for use with chemotherapy using one or more cytotoxic agent (ie., chemotherapeutic), including, but not limited to, cyclophosphamide, taxol, 5-fluorouracil, adriamycin, cisplatinum, methotrexate, cytosine arabinoside, mitomycin C, prednisone, vindesine, carbaplatinum, and vincristine. The cytotoxic agent can also be an antiviral compound which is capable of destroying proliferating cells. For a general discussion of cytotoxic agents used in chemotherapy, see Sathe, M. et al. (1978) Cancer Chemotherapeutic Agents: Handbook of Clinical Data, hereby incorporated by reference. When administered as a combination, the therapeutic agents can be formulated as separate compositions that are given at the same time or different times, or the therapeutic agents can be given as a single composition. The methods of the invention are also particularly suitable for those patients in need of repeated or high doses of chemotherapy and/or radiation therapy.

Any infection to which the immune system responds can be treated according to the methods of the invention. Infections, as used herein, are broadly defined to mean situations when the invasion of a host by an agent is associated with the clinical manifestations of infection including, but not limited to, at least one of the following: abnormal temperature, increased heart rate, abnormal respiratory rate, abnormal white blood cell count, fatigue, chills, muscle ache, pain, dizziness, dehydration, vomiting, diarrhea, organ dysfunction, and sepsis. Such infections may be bacterial, viral, parasitic, or fungal in nature. The method may further comprise combinatorial treatment with other anti-infective agents, such as antibiotics. Viruses susceptible to treatment according to the methods of the invention include, but are not limited to adenoviruses, rhinoviruses, rabies, murine leukemia virus, poxviruses, lentiviruses, retroviruses; including disease-causing viruses such as human immunodeficiency virus, hepatitis A and B viruses, herpes simplex virus, cytomegalovirus, human papilloma virus, coxsackie virus, smallpox, hemorrhagic virus, ebola, and human T-cell-leukemia virus. Bacteria susceptible to treatment include, but are not limited to gram negative bacteria and gram-positive bacteria, including but not limited to Escherichia coli, Staphylococcus aureus, Staphylococcus epidermidis, Streptococcus pneumoniae, Mycobacterium tuberculosis, Neisseria gonorrhoeae, Neisseria meningitis, Bordetalla pertussis, Salmonella thyhimurium, Salmonella choleraesuis, and Enterobacter cloacae, as well as bacterium in the genus Acinetobacter, Actinomyes, Bacilus, Bordetella, Borrelia, Brocella, Clostridium, Coiynebacterium, Campylobacter, Deincoccus, Escherichia, Enterobacter, Enterrococcus, Eubacterium, Flavobacterium, Francisella Glueonobacter, Heliobacter, Intrasporangium, Janthinobacterium, Klebsiella, Kingella, Legionella, Leptospira, Mycobacterium, Moraxella, Neisseria, Oscillospira, Proteus, Psendomonas, Providencia, Rickettsia, Salomonella, Staphylococcus, Shigella, Spirilum, Streptococcus, Treponema, Ureplasma, Vibrio, Wolinella, Wolbachia, Xanthomonas, Yersinis, and Zoogloea Parasitic agents that can be treated by the methods of this aspect of the invention include, but are not limited to Plasmodium, Leishmania, Trypanosomes, Trichomona, and including but not limited to parasitic agents in the phylums Acanthocephela, Nematoda, Neintomorpha, Platylelminthes, Digena, Eucestoda, Turbellaria, Sarcomastigophora and Protozoa including but not limited to species Giardia duodenalis, Cryptosporidium parvum, Cyclospora cayetanenis, Toxoplasma gondii, Trichinella spiralis, Tanenia saginata, Taenia solium, Wuchereria bancrofti, Brugia malay, Brugia timori, Onchocerca vovulus, Loa loa, Dracunculus medinensis, Mansonella streptocera, Mansonella perstans, Mansonella ozzardi, Schistosoma hematobium, Schistosoina mansoni, Schistosoma japonicum, Ascaris lumbricoides, Entrobius vermicularis, Trichuris trichiura, Ancylostoma brasiliense, Ancylostoma duodenale, Necator ameicanus, Strongyloides stercoralis, Capillaria hepatica, Angiostrongylus cantonensis, Fasciola hepatica, Fasciola gigantica, Fasciolopsis buski, Chlonrchis sinensis, Heterophyes heterophyes, Paragonimus westermani, Diphyllobothrium latum, Hyinenolepis nana, Hymenolepis dimunuta, Echinococcus granulosus, Dipylidium caninum, Entamoeba histolytica, Entamoeba coli, Entamoeba hartmanni, Dientamoeba fragilis, Endolimax nana, Lodomoeba butschilii, Blastocystis hominis, Giardia intetinalis, Chilomastix menili, Blantidium coli, Trichomonas vaginalis, Leishmania donovani, Trypanosoma cruzi, Sarcocystis lindemanni, and Babesis argentina. Fungal infections that can be treated by the methods of this aspect of the invention include, but are not limited to fungal meningitis, histoplasmosis, Candida albicans infection, as well as Blastomyces dermatitidis Histotplasma capsulatum, Cryptococcus neoformans, Sporothrix schenckii, Aspergillus fumigatus and Pneumocystis carinii infections.

Angiogenesis-mediated disorders susceptible of treatment by the methods of the invention include solid and blood-borne tumors including but not limited to melanomas, carcinomas, sarcomas, rhabdomyosarcoma, retinoblastoma, Ewing sarcoma, neuroblastoma, osteosarcoma, and leukemia; diabetic retinopathy, rheumatoid arthritis, retinal neovascularization, choroidal neovascularization, macular degeneration, corneal neovascularization, retinopathy of prematurity, corneal graft rejection, neovascular glaucoma, retrolental fibroplasia, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren ulcer, Terrien's marginal degeneration, marginal keratolysis, traum, systemic lupus, polyarteritis, Wegeners sarcoidosis, scleritis, Steven's Johnson disease, radial keratotomy, sickle cell anemia, sarcoidosis, pseudoxanthoma elasticum, Pagets disease, vein occlusion, artery occulsion, carotid obstructive disease, chronic uveitis, chronic vitritis, Lyme's disease, Eales disease, Bechets disease, myopia, optic pits, Stargarts disease, pars planitis, chronic retinal detachment, hyperviscosity syndromes, toxoplasmosis, post-laser complications, abnormal proliferation of fibrovascular tissue, hemangiomas, Osler-Weber-Rendu, acquired immune deficiency syndrome, ocular neovascular disease, osteoarthritis, chronic inflammation, Crohn's disease, ulceritive colitis, psoriasis, atherosclerosis, and pemphigoid. (See U.S. Pat. No. 5,712,291)

Bone disorders susceptible of treatment by the methods of the invention include but are not limited to bone fractures, defects, and disorders resulting in weakened bones such as ostepetrosis, osteoarthritis, rheumatoid arthritis, Paget's disease, osteohalisteresis, osteomalacia, periodontal disease, bone loss resulting from multiple myeloma and other forms of cancer, bone loss resulting from side effects of other medical treatment (such as steroids), age-related loss of bone mass and genetic diseases such as osteopetrosis. The polypeptides of the invention can be used alone or together with other compounds to treat bone disorders.

Immune suppressed illnesses or conditions susceptible of treatment by the methods of the invention include but are not limited to severe combined immune deficiency syndrome, acquired immune deficiency syndrome, and at risk populations including but not limited to malnourished individuals and senior citizens. The proteins of the invention can be used alone or together with other compounds to treat immune suppressed illnesses.

While the invention has been described with reference to a particular embodiment, it will be understood by those skilled in the art that various changes may be made and equivalents may be substituted for elements thereof without departing from the scope of the invention. In addition, many modifications may be made to adapt to a particular situation or material to the teachings of the invention without departing from the essential scope thereof. Therefore, it is intended that the invention not be limited to the particular embodiment disclosed as best mode contemplated for carrying out this invention, but that the invention will include all embodiments falling within the scope of the appended claims.

We claim:

1. A peptide comprising an amino acid sequence selected from the group consisting of SEQ ID 1; SEQ ID 2; SEQ ID 3; SEQ ID 4; and SEQ ID 5.

2. The peptide of claim 1, wherein a threonine residue is glycosylated.

3. The peptide of claim 1, wherein a threonine is substituted with an amino acid selected from the group consisting of serine; lysine; glutamic acid; asparagine; aspartic acid; and glutamine.

4. The peptide of claim 2, wherein a threonine is substituted with an amino acid selected from the group consisting of serine; lysine; glutamic acid, asparagine; aspartic acid; and glutamine.

5. The peptide of claim 1, wherein an asparagine is substituted with an amino acid selected from the group consisting of aspartic acid; glutamic acid; and glycine.

6. The peptide of claim 1, wherein a lysine is substituted with an amino acid selected from the group consisting of aspartic acid; glutamic acid; alanine; asparagine; glutamine; and arginine.

7. The peptide of claim 1, wherein an alanine is substituted with an amino acid selected from the group consisting of leucine; phenylalanine; isoleucine; tryptophan; asparagine; glutamine; and valine.

8. The peptide of claim 1, wherein a leucine is substituted with an amino acid selected from the group consisting of alanine; phenylalanine; isoleucine; tryptophan; tyrosine; and valine.

9. The peptide of claim 1, wherein a glutamic acid is substituted with an amino acid selected from the group consisting of lysine; asparagine; arginine; aspartic acid; and glutamine.

10. The peptide of claim 1, wherein a valine is substituted with an amino acid selected from the group consisting of alanine; phenylalanine; isoleucine; tryptophan; tyrosine; and leucine.

11. The peptide of claim 1, wherein a hexose is attached to the threonine.

12. The peptide of claim 2, wherein a hexosamine is attached to the threonine.

13. A method for treating immunosuppressive disease in an animal comprised of administering an effective dose of an immunoactive substance comprised of an immunomodulatory protein mimetic including a peptide selected from the group consisting of SEQ ID 1; SEQ ID 2; SEQ ID 3; SEQ ID 4; and SEQ ID 5, wherein a threonine is glycosylated; and wherein immune system activity is increased.

14. The method of claim 13, wherein the immunosuppressive disease is selected from a group consisting of cancer, AIDS, and influenza.

15. The method of claim 13, wherein phagocytosis is increased.

16. The method of claim 13, wherein the animal is a human.

17. A laboratory kit useful in increasing phagocytic activity of immune cells comprised of a protein mimetic including a peptide selected from the group consisting of SEQ ID 1; SEQ ID 2; SEQ ID 3; SEQ ID 4; and SEQ ID 5, wherein a threonine residue is glycosylated.

18. The laboratory kit of claim 17, wherein the phagocytic cells are monocytes.

19. The laboratory kit of claim 17, wherein the phagocytic cells are macrophages.


SEQUENCE LISTING (7 sequences)

SEQ ID NO: 1 - protein, 69 aa, Artificial
  Synthetic; GC-MAF
GSRVKALEEKVKALEEKVKALGNATPTELAKKKWEELKKKIEELGNATPTEVKKVEEEVKKLEEEIKKL

SEQ ID NO: 2 - protein, 69 aa, Artificial
  Synthetic; GC-MAF
GSRVKALEEKVKALEEKVKALGGGGRIEELKKKWEELKKKIEELGNATPTEVKKVEEEVKKLEEEIKKL

SEQ ID NO: 3 - protein, 67 aa, Artificial
  Synthetic; GC-MAF
GSRVKALEEKVKALEEKVKALGNATPTELAKKKWEELKKKIEELGGGGEVKKVEEEVKKLEEEIKKL

SEQ ID NO: 4 - protein, 10 aa, Artificial
  Synthetic; GC-MAF
GNATPTELAK

SEQ ID NO: 5 - protein, 10 aa, Artificial
  Synthetic; GC-MAF
GNATPTEVKK

SEQ ID NO: 6 - protein, 10 aa, Artificial
  Synthetic
  MISC_FEATURE (1)..(1): X stands for G, P, A, N, or S, (or is absent).
  MISC_FEATURE (2)..(2): X stands for N, D, E, or G (or is absent).
  MISC_FEATURE (3)..(3): X stands for A, G, L, V, or F (or is absent).
  MISC_FEATURE (4)..(4): X stands for T, N, S, K, E, Q, or D (or is absent).
  MISC_FEATURE (5)..(5): X stands for P or G (or is absent).
  MISC_FEATURE (6)..(6): X stands for T, S, K, E, D, N, or Q (or is absent).
  MISC_FEATURE (7)..(7): X stands for E, N, Q, D, K, or R (or is absent).
  MISC_FEATURE (8)..(8): X stands for L, A, V, I, F, W, or Y (or is absent).
  MISC_FEATURE (9)..(9): X stands for A, L, V, I, F, W, Q, or N (or is absent).
  MISC_FEATURE (10)..(10): X stands for K, R, D, E, N, or Q (or is absent).
XXXXXXXXXX

SEQ ID NO: 7 - protein, 10 aa, Artificial
  Synthetic, Loop 2 Sequences
  MISC_FEATURE (1)..(1): X stands for G, P, A, N, or S (or is absent).
  MISC_FEATURE (2)..(2): X stands for N, E, D, or G (or is absent).
  MISC_FEATURE (3)..(3): X stands for A, G, L, V, or F (or is absent).
  MISC_FEATURE (4)..(4): X stands for T, N, S, K, E, D, or Q (or is absent).
  MISC_FEATURE (5)..(5): X stands for P or G (or is absent).
  MISC_FEATURE (6)..(6): X stands for T, S, K, E, N, D, or Q (or is absent).
  MISC_FEATURE (7)..(7): X stands for E, N, Q, D, K, or R (or is absent).
  MISC_FEATURE (8)..(8): X stands for V, A, L, I, F, W, or Y (or is absent).
  MISC_FEATURE (9)..(9): X stands for K, R, D, E, N, Q, or A (or is absent).
  MISC_FEATURE (10)..(10): X stands for K, R, D, E, N, or Q (or is absent).
XXXXXXXXXX